(19) United States
(12) Patent Application Publication
(10) Pub. No.: US 2010/0224869 A1
(43) Pub. Date: Sep. 9, 2010

(54) ORGANIC SEMICONDUCTOR MATERIAL, ORGANIC SEMICONDUCTOR STRUCTURE AND ORGANIC SEMICONDUCTOR APPARATUS

(75) Inventors: Ken Tomino, Shinjuku-ku (JP); Shigeru Sugawara, Shinjuku-ku (JP); Hiroki Maeda, Shinjuku-ku (JP); Masanao Matsuoka, Shinjuku-ku (JP)

Correspondence Address: BURR & BROWN, PO BOX 7068, SYRACUSE, NY, 13261-7068, US

(73) Assignee: Dai Nippon Printing Co., Ltd., Shinjuku-ku (JP)

(21) Appl. No.: 12/782,046

(22) Filed: May 18, 2010

Related U.S. Application Data
(63) Continuation of application No. 11/445,919, filed on Jun. 2, 2006, now abandoned.

(30) Foreign Application Priority Data
Jun. 3, 2005 (JP) 2005-163553

(51) Int. Cl. H01L 51/10; C07D 409/14

(52) U.S. Cl. 257/40; 549/59; 257/E51.006

(57) ABSTRACT

The present invention is directed to the provision of a liquid crystalline organic semiconductor material, which is highly stable under a film forming environment and, at the same time, can easily form a film, for example, by coating. The liquid crystalline organic semiconductor material comprises: a thiophene skeleton comprising 3 to 6 thiophenes linearly connected to each other; and an identical alkyl group having 1 to 20 carbon atoms located on both sides of the thiophene skeleton, wherein acetylene skeletons each have been introduced into between the thiophene skeleton and the alkyl group, or acetylene skeletons have been introduced symmetrically into the thiophene skeleton.

### CROSS-REFERENCE TO RELATED APPLICATIONS

This application is a continuation of U.S. application Ser. No. 11/445,919, filed Jun. 2, 2006, and claims the benefit under 35 USC §119(a)-(d) of Japanese Patent Application No. 2005-163553, filed Jun. 3, 2005, the entireties of which are incorporated herein by reference.

### FIELD OF THE INVENTION

The present invention relates to a liquid crystalline organic semiconductor material, which is highly stable under a film forming environment and, at the same time, can easily form a film, for example, by coating, an organic semiconductor structure and an organic semiconductor device.

### BACKGROUND OF THE INVENTION

Attention has recently been drawn to studies on organic semiconductor structures using an organic semiconductor material, and application of organic semiconductor structures to various devices has been expected. Devices utilizable, for example, in large-area flexible display devices, for example, thin-film transistors (also known as “organic TFTs”), luminescent elements, and solar cells are being studied for such application.

In order to utilize organic semiconductor structures on a practical level, the organic semiconductor layer formed of an organic semiconductor material should exhibit stable charge mobility in a wide service temperature range, and, at the same time, even thin film should be easily formed in a wide area. In particular, properties satisfying the following requirements are desired: the formation of a film by coating rather than film formation by conventional techniques such as vapor deposition is possible; properties in a film formation environment are stable; and stable high charge mobility can be exhibited in a wide service temperature range including room temperature (about −40 to +90° C.).

Regarding prior art documents relevant to the present invention, for example, non-patent document 1 describes an oligothiophene compound represented by the following chemical formula 3 as a nonlinear optical material. Non-patent document 2 describes a non-liquid crystalline oligothiophene compound represented by the following chemical formula 4. Non-patent document 3 describes an oligothiophene compound (whether or not this compound is liquid crystalline is unknown) synthesized as a metal complex precursor represented by the following chemical formula 5.

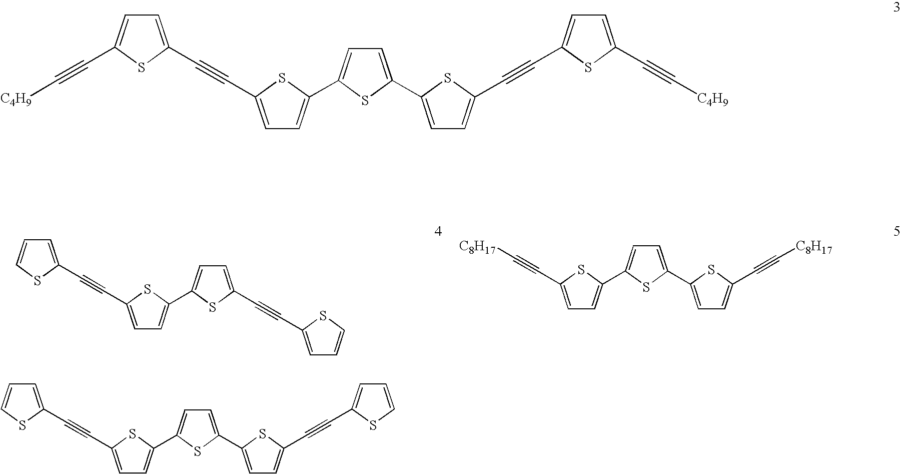

Non-patent document 1: H. Zhang, T. Ikeda, et al., Adv. Mater., vol. 12, No. 18, p. 1336 to 1339 (2000)

Non-patent document 2: M. Melucci, G. Barbarella, et al., J. Org. Chem., vol. 69, p. 4821-4828 (2004)

Non-patent document 3: T. S. Jung, J. H. Kim, et al., J. Organometal. Chem., vol. 599, No. 2, p. 232 to 237 (2000).

### SUMMARY OF THE INVENTION

### Problems to be Solved by the Invention

Properties desired to be possessed by organic semiconductor materials for forming organic TFTs utilizable on a practical level, for example, in large-area flexible display devices include that the materials are soluble in solvents and can easily be brought to coating liquids, the properties of the materials are stable under a film formation environment, and films having stable charge mobility in a wide service temperature range including room temperature can be formed. The development of such organic semiconductor materials has been expected.

The present invention has been made with a view to meeting the above demand, and an object of the present invention is to provide a novel liquid crystalline organic semiconductor material that is highly stable under a film formation environment and, at the same time, can easily be brought to a film, for example, by coating. Another object of the present invention is to provide an organic semiconductor structure and an organic semiconductor device comprising an organic semiconductor layer formed of this organic semiconductor material.

### Means for Solving the Problems

The above object of the present invention is attained by an organic semiconductor material comprising: a thiophene skeleton comprising 3 to 6 thiophenes linearly connected to each other; and an identical alkyl group having 1 to 20 carbon atoms located on both sides of said thiophene skeleton, characterized in that acetylene skeletons have been introduced into between said thiophene skeleton and said alkyl group, or acetylene skeletons have been introduced symmetrically into said thiophene skeleton.

(i) In the organic semiconductor material according to this invention, since an identical alkyl group having 1 to 20 carbon atoms is located on both sides of a straight chain thiophene skeleton, the organic semiconductor material is liquid crystalline and is soluble in solvents. Coating liquids prepared by dissolving this organic semiconductor material in a solvent can easily realize the formation of an organic semiconductor layer utilizable, for example, in large-area flexible display devices. (ii) Since weakly electron-withdrawing acetylene skeletons (acetylene skeletons introduced into the oligothiophene skeleton behave as a weakly electron withdrawing group) have been each introduced into between the electron-donating thiophene skeleton and the alkyl group, or the weakly electron-withdrawing acetylene skeletons have been introduced symmetrically into the electron-donating thiophene skeleton, in the organic semiconductor material according to the present invention, advantageously, π electrons can be delocalized, lifting of HOMO (highest occupied molecular orbital) can be suppressed, and LUMO (lowest unoccupied molecular orbital) can be lowered. As a result, the above chemical structure can advantageously narrow the band gap of the organic semiconductor material according to the present invention and further can suppress an increase in ionization potential. In particular, the suppression of the increase in ionization potential can suppress oxidation under a film formation environment. Therefore, an organic semiconductor layer, which is less likely to undergo oxidation and the like and is stable, can be formed by forming the organic semiconductor layer using this organic semiconductor material. (iii) The liquid crystalline organic semiconductor material according to the present invention has an acetylene skeleton and thus has a lowered phase transition temperature. Accordingly, the formation of an organic semiconductor layer by coating is easier.

The above organic semiconductor material is characterized by being represented by chemical formula 1 wherein R1 and R2 represent an identical alkyl group having 1 to 20 carbon atoms and n1 is 3 to 6:

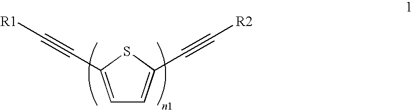

The above organic semiconductor material is characterized by being represented by chemical formula 2 wherein R3 and R4 represent an identical alkyl group having 1 to 20 carbon atoms and n2 is 1 to 4:

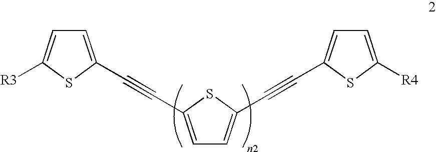

The organic semiconductor structure according to the present invention is attained by an organic semiconductor structure characterized by comprising an organic semiconductor layer formed of the above organic semiconductor material according to the present invention, said organic semiconductor layer having a smectic liquid crystal phase or a crystal phase at least in a room temperature region.

According to the present invention, since the organic semiconductor material according to the present invention is a liquid crystalline material having excellent solubility in solvents, the formation of an organic semiconductor layer by using a coating liquid comprising this organic semiconductor material can easily realize the formation of an organic semiconductor structure utilizable, for example, in large-area flexible display devices. Further, since the organic semiconductor layer formed of the organic semiconductor material according to the present invention has a smectic liquid crystal phase or a crystal phase at least in a room temperature region, for example, when a coating liquid containing the organic semiconductor material is heated to bring the phase to an isotropic phase or a liquid crystal phase and, in this heated state, is coated followed by cooling to room temperature, a smectic liquid crystal phase or a crystal phase, in which a thiophene skeleton and an alkyl chain part are arranged in alignment relationship is formed and, consequently, stable charge mobility can be realized at least in a room temperature region.

The above object of the present invention can be attained by an organic semiconductor device characterized by comprising at least a substrate, a gate electrode, a gate insulating layer, an organic semiconductor layer, a drain electrode, and a source electrode, said organic semiconductor layer being formed of the above organic semiconductor material according to the present invention. According to this invention, since the organic semiconductor layer is formed using a liquid crystalline organic semiconductor material which is highly stable under a film formation environment and, at the same time, can be easily brought to a film, for example, by coating, an organic semiconductor device utilizable, for example, in large-area flexible display devices can easily be formed.

Further, according to the present invention, there is also provided use of the above organic semiconductor structure, as an organic transistor, an organic EL element, an organic electronic device, or an organic solar cell.

Since the organic semiconductor material according to the present invention is liquid crystalline and, at the same time, is soluble in solvents, coating liquids prepared by dissolving such organic semiconductor materials in solvents can easily realize the formation of an organic semiconductor layer utilizable, for example, in large-area flexible display devices. By virtue of this chemical structure, advantageously, π electrons can be delocalized, lifting of HOMO can be suppressed, and LUMO can be lowered. As a result, advantageously, the band gap of the organic semiconductor material can be narrowed, and, at the same time, an increase in ionization potential can be suppressed. In particular, a stable organic semiconductor layer, which is less likely to undergo oxidation and the like, can be formed. Further, since the organic semiconductor material has an acetylene skeleton, the phase transition temperature of the liquid crystalline organic semiconductor material is lowered, and the formation of the organic semiconductor layer by coating becomes easier.

In the organic semiconductor structure according to the present invention, an organic semiconductor structure utilizable, for example, in large-area flexible display devices can easily be formed. Further, when a coating liquid containing the organic semiconductor material is heated to bring the phase to an isotropic phase or a liquid crystal phase and, in this heated state, is coated followed by cooling to room temperature, a smectic liquid crystal phase or a crystal phase, in which a thiophene skeleton and an alkyl chain part are arranged in alignment relationship is formed and, consequently, stable charge mobility can be realized.

Further, the organic semiconductor device according to the present invention can be used in devices utilizable, for example, in large-area flexible display devices, for example, thin-film transistors, luminescent elements, and solar cells.

### BRIEF DESCRIPTION OF THE DRAWINGS

FIG. 1 is a cross-sectional view showing one embodiment of the organic semiconductor device according to the present invention;

FIG. 2 is a diagram showing the results of observation of texture by a polarizing microscope and a heating stage using a glass cell into which 8T-yne-TTP-yne-T8 has been poured;

FIG. 3 is a diagram showing the results of observation of texture by a polarizing microscope and a heating stage using a glass cell into which 8T-yne-TTP-yne-T8 has been poured;

FIG. 4 is a diagram showing the results of observation of texture by a polarizing microscope and a heating stage using a glass cell into which 8-yne-QT-yne-8 has been poured;

### DESCRIPTION OF REFERENCE CHARACTERS

101: organic semiconductor device,
11: substrate,
12: gate electrode,
13: gate insulating layer,
14: polymeric organic semiconductor layer,
15: drain electrode, and
16: source electrode.

### DETAILED DESCRIPTION OF THE INVENTION

The organic semiconductor material, organic semiconductor structure, and organic semiconductor device according to the present invention will be described.

### (Organic Semiconductor Material)

The organic semiconductor material according to the present invention comprises a thiophene skeleton comprising 3 to 6 thiophenes linearly connected to each other and an identical alkyl group having 1 to 20 carbon atoms (number of carbon atoms being hereinafter represented by “C”), that is, a C1 to C20 identical alkyl group, located on both sides of the thiophene skeleton, characterized in that acetylene skeletons have been each introduced into between the thiophene skeleton and the alkyl group (this being referred to as “skeleton end introduction type”), or acetylene skeletons have been introduced symmetrically into the thiophene skeleton (this being referred to as “skeleton internal introduction type”). In the present specification, the organic semiconductor material is often referred to as “oligothiophene compound.”

The skeleton end introduction-type organic semiconductor material is an oligothiophene compound comprising a thiophene skeleton comprising 3 to 6 thiophenes linearly connected to each other and an acetylene skeleton having a C1 to C20 identical alkyl group on both ends of the thiophene skeleton. In other words, as described above, the skeleton end introduction-type organic semiconductor material is an oligothiophene compound comprising acetylene skeletons each introduced into between the thiophene skeleton and the alkyl group. Specifically, the skeleton end introduction-type organic semiconductor material is represented by chemical formula 1. In chemical formula 1, R1 and R2 represent a C1 to C20 identical alkyl group and may be a straight chain or branched chain type. A straight chain alkyl group is preferred. n1 is 3 to 6.

The skeleton internal introduction-type organic semiconductor material is an oligothiophene compound comprising a thiophene skeleton comprising 3 to 6 thiophenes linearly connected to each other and an identical alkyl group having a C1 to C20 identical alkyl group located on both sides of the thiophene skeleton, wherein acetylene skeletons have been introduced symmetrically into the thiophene skeleton. Specifically, the skeleton internal introduction-type organic semiconductor material is represented by chemical formula 2. In chemical formula 2, R3 and R4 represent a C1 to C20 identical alkyl group and may be a straight chain or branched chain type. A straight chain alkyl group is preferred. n2 is 1 to 4.

In the organic semiconductor materials represented by chemical formulae 1 and 2, the alkyl groups are bilaterally symmetrical due to the nature of the production. Accordingly, an identical alkyl group is present on both sides in the thiophene skeleton. The number of carbon atoms of the alkyl group is preferably in the range of C1 to C16 from the viewpoints of liquid crystallinity and solubility in solvents.

As is apparent from chemical formulae 1 and 2, since a C1 to C20 identical alkyl group is located on both sides of the straight chain thiophene skeleton, the organic semiconductor materials according to the present invention is liquid crystalline and, at the same time, is soluble in solvents. An organic semiconductor layer utilizable, for example, in large-area flexible display devices can easily be formed by dissolving the organic semiconductor material in a solvent such as toluene, xylene, mesitylene, tetralin, monochlorobenzene, or o-dichlorobenzene to prepare a coating liquid and then coating the coating liquid onto a predetermined base material such as a plastic substrate or a glass substrate optionally with various films formed thereon. In particular, when a coating liquid containing the organic semiconductor material according to the present invention is heated to bring the phase to an isotropic phase or a liquid crystal phase and, in this heated state, is coated followed by cooling, in the oligothiophene compound (organic semiconductor material) according to the present invention, a core part comprising a straight chain thiophene skeleton part and an alkyl chain part are arranged in alignment relationship and, consequently, stable charge mobility can be realized, for example, by hopping conduction in the thiophene skeleton part.

Further, in the organic semiconductor material according to the present invention, as is apparent from the above chemical formulae 1 and 2, weakly electron-withdrawing acetylene skeletons (acetylene skeletons introduced into the oligothiophene skeleton behave as a weakly electron withdrawing group) each have been introduced into between the electron-donating thiophene skeleton and the alkyl group, or the weakly electron-withdrawing acetylene skeletons have been introduced symmetrically into the electron-donating thiophene skeleton, advantageously. Accordingly, π electrons can be delocalized within the oligothiophene compound, lifting of HOMO (highest occupied molecular orbital) can be suppressed and LUMO can be reduced. Thus, the introduction of the acetylene skeleton into the oligothiophene compound is advantageous for the narrowing of the oligothiophene compound, and, at the same time, can suppress an increase in the ionization potential. In particular, the suppression of the increase in ionization potential can suppress oxidation under an organic semiconductor layer formation environment (for example, in the atmosphere). Therefore, an organic semiconductor layer, which is less likely to undergo oxidation and the like and is stable, can be formed by forming the organic semiconductor layer using this organic semiconductor material. The HOMO and LUMO values of the oligothiophene compounds as the organic semiconductor materials according to the present invention are determined by the calculation of DFT (B3LYP/6-31G(d) method). For example, 5,5″-dimethyl-2,2′:5,2″-terthiophene, which is not the organic semiconductor material according to the present invention, is HOMO=−4.97 eV and LUMO=−1.53 eV. On the other hand, (a) 2,5-bis(5-methyl-2-thienylethynyl)-thiophene (1T-yne-T-yne-T1), which is an oligothiophene compound represented by formula 2 wherein R3 and R4 represent a methyl group and n2 is 1, is HOMO=−5.00 eV and LUMO=−1.92 eV, and (b) 5,5″-bis(methyl-2-yne)-2,2′:5′2″-terthiophene (1-yne-TTP-yne-1), which is an oligothiophene compound represented by formula 1 wherein R1 and R2 represent a methyl group and n1 is 3, is HOMO=−4.96 eV and LUMO=−1.92 eV. Thus, the HOMO and LUMO values of the oligothiophene compounds as the organic semiconductor materials according to the present invention are not very influenced by the position of the introduction of the weakly electron-withdrawing acetylene skeletons, that is, is by whether the weakly electron-withdrawing acetylene skeletons each have been introduced into between the thiophene skeleton and the alkyl group, or the weakly electron-withdrawing acetylene skeletons have been introduced symmetrically into the thiophene skeleton.

Further, in the organic semiconductor materials according to the present invention, an acetylene skeleton is contained in a bilaterally symmetrical form on both sides of the thiophene skeleton or within the thiophene skeleton. Therefore, as compared with the acetylene skeleton-free compound and the compound containing only one acetylene skeleton, the phase transition temperature of the liquid crystalline organic semiconductor material is lowered. As a result, the temperature at which the organic semiconductor material can be brought to an isotropic phase state or a liquid crystal phase state by heating can be lowered, and, thus, subsequent formation of the organic semiconductor layer by coating and cooling becomes easier.

### (Organic Semiconductor Structure)

The organic semiconductor structure according to the present invention comprises an organic semiconductor layer formed of the above organic semiconductor material. The organic semiconductor layer has a smectic liquid crystal phase or a crystal phase at least in the room temperature region. In the present invention, the room temperature region refers to a temperature range of −40° C. to 90° C. which is a common service temperature range of semiconductor elements such as organic TFTs.

According to DSC (differential scanning calorimeter, DSC204u-Sensor manufactured by NETZSCH) measurement, for example, 5,5″-bis(decyl-2-yne)-2,2′:5′″,2″-terthiophene (hereinafter referred to also as “8-yne-TTP-yne-8”), which is an oligothiophene compound represented by chemical formula 6, has a phase transition temperature of crystal phase/30.6° C./smectic G phase (SmG phase)/65.8° C./isotropic phase, 5,5′-bis(decyl-2-yne)-2,2′:5′,2″:5″,2′″-quaterthiophene (hereinafter referred to also as “8-yne-QT-yne-8”), which is an oligothiophene compound represented by chemical formula 7, has a phase transition temperature of crystal phase/101.2° C./SmG phase/164.7° C./isotropic phase, 5,5′-bis(5-octyl-2-thienylethynyl)-2,2′-bithiophene (hereinafter referred to also as “8T-yne-TT-yne-T8”), which is an oligothiophene compound represented by chemical formula 8, has a phase transition temperature of crystal phase/88.9° C./smectic X1 phase (SmX1 phase)/94.3° C./isotropic phase, and 5,5″-bis(5-octyl-2-thienylethynyl)-2,2′:5′,2″-terthiophene (hereinafter referred to also as “8T-yne-TTP-yne-T8”), which is an oligothiophene compound represented by chemical formula 9, has a phase transition temperature of crystal phase/77.2° C./SmX2 phase/111.2° C./SmX1 phase/136.5° C./nematic phase/159.4° C./isotropic phase.

The temperature indicated between the phases refers to the phase transition temperature between the phase indicated on the left side and the phase indicated on the right side. For example, “crystal phase/30.6° C./SmG phase” means that the phase transition temperature between the crystal phase and the SmG phase is 30.6° C.

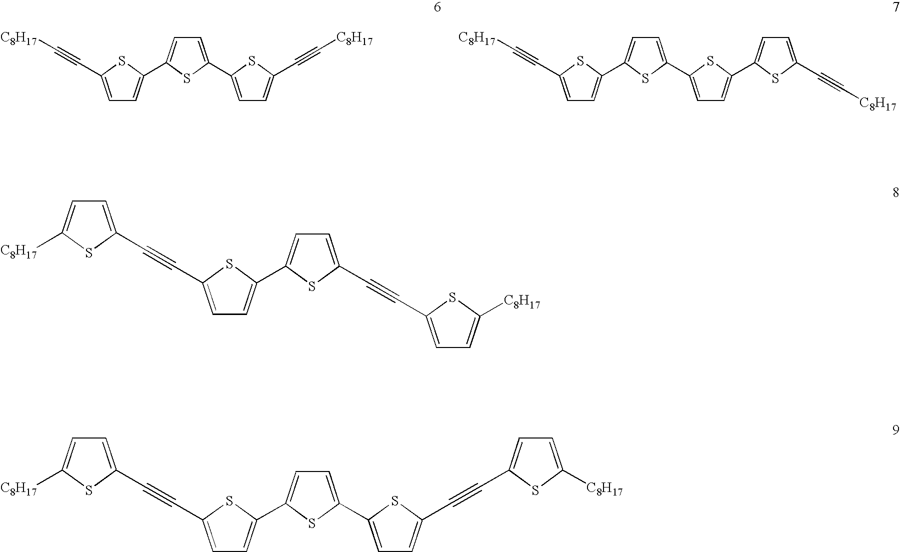

When a coating liquid containing the organic semiconductor material is heated to a temperature above at least the crystallization temperature to bring the phase to an isotropic phase or a liquid crystal phase and, in this heated state, is coated on a substrate followed by cooling to room temperature, a smectic liquid crystal phase or a crystal phase, in which a thiophene skeleton and an alkyl chain part of each oligothiophene compound are arranged in alignment relationship is formed and, consequently, stable charge mobility can be realized at least in a room temperature region (−40° C. to 90° C.). In this case, various coating methods and printing methods can be applied for coating.

Alignment in coating the organic semiconductor material onto a substrate can be carried out by coating the organic semiconductor material onto a liquid crystal aligning layer formed of a polyimide material, or by coating the organic semiconductor material onto a liquid crystal aligning layer formed of a cured resin having very small concaves and convexes on its surface.

A first embodiment of the organic semiconductor structure according to the present invention comprises a substrate, a liquid crystal aligning layer, and an organic semiconductor layer stacked in that order. A second embodiment of the organic semiconductor structure according to the present invention comprises a substrate, an organic semiconductor layer, and a liquid crystal aligning layer stacked in that order. A third embodiment of the organic semiconductor structure according to the present invention comprises a substrate, a liquid crystal aligning layer, an organic semiconductor layer, and a liquid crystal aligning layer stacked in that order. In the present invention, a high level of alignment can be imparted to the organic semiconductor layer by forming the organic semiconductor layer in contact with the liquid crystal aligning layer.

As described above, in the organic semiconductor structure according to the present invention, when a coating liquid containing the organic semiconductor material is heated to bring the phase to an isotropic phase or a liquid crystal phase and, in this heated state, is coated followed by cooling to room temperature, a smectic liquid crystal phase or a crystal phase, in which a thiophene skeleton and an alkyl chain part are arranged in alignment relationship is formed and, consequently, stable charge mobility can be realized at least in the room temperature region (−40° C. to 90° C.). As a result, application to a semiconductor layer, for example, to thin-film transistors and field-effect transistors utilizable, for example, in large-area flexible display devices can be expected.

### (Organic Semiconductor Device)

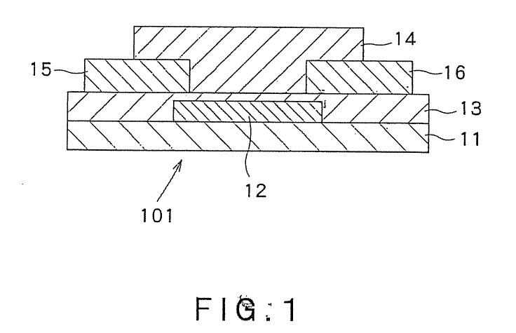

An organic semiconductor device 101 according to the present invention, for example, as shown in FIG. 1, comprises at least a substrate 11, a gate electrode 12, a gate insulating layer 13, an organic semiconductor layer 14, a drain electrode 15, and a source electrode 16. In this organic semiconductor device 101, the organic semiconductor layer 14 is formed of the organic semiconductor material constituting the organic semiconductor structure according to the present invention.

Examples of the construction include a reversed stagger structure (not shown) comprising a substrate 11 and a gate electrode 12, a gate insulating layer 13, an aligned organic semiconductor layer 14, a drain electrode 15 and a source electrode 16, and a protective film 17 provided in that order on the substrate 11, or a coplanar structure (see FIG. 1) comprising a substrate 11 and a gate electrode 12, a gate insulating layer 13, a drain electrode 15 and a source electrode 16, an organic semiconductor layer 14, and a protective film (not shown) provided in that order on the substrate 11. The organic semiconductor device 101 having the above construction is operated in either an storage state or a deficiency state depending upon the polarity of the voltage applied to the gate electrode 12. Members for constituting the organic semiconductor device will be described in detail.

### (Substrate)

The substrate 11 may be selected form a wide range of insulating materials. Examples of such materials include inorganic materials such as glasses and alumina sinters, polyimide films, polyester films, polyethylene films, polyphenylene sulfide films, poly-p-xylene films and other various insulating materials. The use of a film or sheet substrate formed of a polymer compound is very useful because a lightweight and flexible organic semiconductor device can be prepared. The thickness of the substrate 11 applied in the present invention is about 25 μm to 1.5 mm.

### (Gate Electrode)

The gate electrode 12 is preferably an electrode formed of an organic material such as polyaniline or polythiophene, or an electrode formed by coating an electrically conductive ink. These electrodes can be formed by coating an organic material or an electrically conductive ink and thus is advantageous in that the electrode formation process is very simple. Specific methods usable for the coating include spin coating, casting, pulling-up, and transfer and ink jet methods.

When a metal film is formed as the electrode, a conventional vacuum film formation method may be used for the metal film formation. Specifically, a mask film formation method or a photolithographic method may be used. In this case, materials usable for electrode formation include metals such as gold, platinum, chromium, palladium, aluminum, indium, molybdenum, and nickel, alloys using these metals, and inorganic materials such as polysilicon, amorphous silicone, tin oxide, indium oxide, and indium tin oxide (ITO). These materials may be used in a combination of two or more.

The film thickness of the gate electrode is preferably about 50 to 1000 nm although the film thickness varies depending upon the electric conductivity of the material for electrode. The lower limit of the thickness of the gate electrode varies depending upon the electric conductivity of the electrode material and the adhesive strength between the gate electrode and the underlying substrate. The upper limit of the thickness of the gate electrode should be such that, when a gate insulating layer and a source-drain electrode pair, which will be described later, are provided, the level difference part between the underlying substrate and the gate electrode is satisfactorily covered for insulation by the gate insulating layer and, at the same time, an electrode pattern formed thereon is not broken. In particular, when a flexible substrate is used, the balance of stress should be taken into consideration.

### (Gate Insulating Layer)

As with the gate electrode 12, the gate insulating layer 13 is preferably formed by coating an organic material. Organic materials usable herein include polychloropyrene, polyethylene terephthalate, polyoxymethylene, polyvinyl chloride, polyvinylidene fluoride, cyanoethylpullulan, polymethyl methacrylate, polysulfone, polycarbonate, and polyimide. Specific examples of methods usable for coating include spin coating, casting, pulling-up, and transfer and ink jet methods. A conventional pattern process such as CVD may also be used. In this case, inorganic materials such as SiO₂, SiNx, and Al₂O₃ are preferred. These materials may be used in a combination of two or more.

Since the charge mobility of the organic semiconductor device depends upon the field strength, the thickness of the gate insulating layer is preferably about 50 to 300 nm. In this case, the withstand voltage is preferably not less than 2 MV/cm.

### (Drain Electrode and Source Electrode)

The drain electrode 15 and the source electrode 16 are preferably formed of a metal having a large work function. The reason for this is that, in the liquid crystalline organic semiconductor material according to the present invention, since carriers for transferring charges are holes, these electrodes should be in ohmic contact with the organic semiconductor layer 14. The work function referred to herein is an electric potential difference necessary for withdrawing electrons in the solid to the outside of the solid and is defined as a difference in energy between a vacuum level and a Fermi level. The work function is preferably about 4.6 to 5.2 eV. Such materials include gold, platinum, and transparent electrically conductive films (for example, indium tin oxide and indium zinc oxide). The transparent electrically conductive film may be formed by sputtering or electron beam (EB) vapor deposition. The thickness of the drain electrode 15 and the source electrode 16 applied in the present invention is about 50 nm.

### (Organic Semiconductor Layer)

The organic semiconductor layer 14 is a layer formed of the organic semiconductor material according to the present invention. In the organic semiconductor layer 14, a smectic liquid crystal phase or a crystal phase, in which a thiophene skeleton and an alkyl chain part are arranged in alignment relationship, is exhibited at least in a temperature range including room temperature. Thus, a characteristic effect that an even and large-area organic semiconductor layer can be formed, can be attained.

When the organic semiconductor material forming face is a gate insulating layer or a substrate, an aligning film can be integrated with the gate insulating layer or the substrate by subjecting the gate insulating layer or the substrate to rubbing treatment.

### (Interlayer Insulating Layer)

An interlayer insulating layer is preferably provided in the organic semiconductor device 101. In forming the drain electrode 15 and the source electrode 16 on the gate insulating layer 13, the interlayer insulating layer is formed to prevent the contamination of the surface of the gate electrode 12. Accordingly, the interlayer insulating layer is formed on the gate insulating layer 13 before the formation of the drain electrode 15 and the source electrode 16. After the formation of the drain electrode 15 and the source electrode 16, the interlayer insulating layer in its part located above the channel region is completely or partly removed. The interlayer insulating layer region to be removed is preferably equal to the size of the gate electrode 12.

Materials usable for the interlayer insulating layer include inorganic material such as SiO₂, SiNx, and Al₂O₃ and organic materials such as polychloropyrene, polyethylene terephthalate, polyoxymethylene, polyvinyl chloride, polyvinylidene fluoride, cyanoethylpullulan, polymethyl methacrylate, polysulfone, polycarbonate, and polyimide.

### Other Embodiments of Organic Semiconductor Device

Examples of the construction of the organic semiconductor device according to the present invention include (i) substrate/gate electrode/gate insulating layer (which functions also as liquid crystal aligning layer)/source-drain electrode/organic semiconductor layer (/protective layer), (ii) substrate/gate electrode/gate insulating layer/source-drain electrode/liquid crystal aligning layer/organic semiconductor layer (/protective layer), (iii) substrate/gate electrode/gate insulating layer (which functions also as liquid crystal aligning layer)/organic semiconductor layer/source-drain electrode/(protective layer), (iv) substrate/gate electrode/gate insulating layer (which functions also as liquid crystal aligning layer)/organic semiconductor layer/substrate with source-drain electrode patterned therein (which functions also as protective layer), (v) substrate/source-drain electrode/organic semiconductor layer/gate insulating layer (which functions also as liquid crystal aligning layer)/gate electrode/substrate (which functions also as protective layer), (vi) substrate (which functions also as aligning layer)/source-drain electrode/organic semiconductor layer/gate insulating layer/gate electrode/substrate (which functions also as protective layer), or (vii) substrate/gate electrode/gate insulating layer/source-drain electrode/organic semiconductor layer/substrate (which functions also as aligning layer).

In the organic semiconductor device, the organic semiconductor layer can easily be formed by coating using the organic semiconductor material according to the present invention.

### EXAMPLES

The following Examples further illustrate the present invention.

### Example 1

In Example 1, an organic semiconductor material represented by chemical formula 2 wherein R3 and R4 represent a C8 identical straight chain alkyl group and n2 is 1 to 3, was prepared.

### Synthesis of 2-octylthiophene

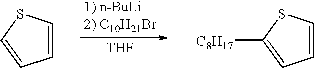

Thiophene (59.9 g, 0.713 mol) and dehydrated tetrahydrofuran (hereinafter referred to as “THF”) (200 ml) were placed in a 1000-ml three-necked flask equipped with a 200-ml dropping funnel and a reflux tube. The solution was cooled to −78° C., and a solution (200 ml) of n-butyllithium (2.6 M) in n-hexane was added dropwise to the cooled solution over a period of about one hr. After the completion of the dropwise addition, the mixture was stirred at −78° C. for about one hr. Thereafter, the reaction temperature was raised to room temperature. At that temperature, the mixture was again stirred for one hr, and 1-bromooctane (91.8 g, 0.475 mol) was added dropwise thereto at 0° C. over a period of about one hr. After the completion of the dropwise addition, the reaction temperature was raised to room temperature, and, at that temperature, the mixture was stirred overnight. After the completion of the reaction, water (200 ml) was added, and the organic layer was extracted with diethyl ether, was dried over sodium sulfate, and was applied to column chromatography (n-hexane) to give an objective compound 2-octylthiophene as a yellow liquid (99.9 g, yield 97.8%). An NMR spectrum of the compound thus obtained was measured at room temperature with an NMR spectrometer (model JNM-LA400W, manufactured by Japan Electric Optical Laboratory; the same apparatus was also used in the following NMR measurement). ¹H-NMR (CDCl₃, TMS/ppm): 0.88 (t, 3H, J=6.83 Hz), 1.28 (m, 10H), 1.67 (m, 2H), 2.81 (t, 2H, J=7.32 Hz), 6.77 (dd, 1H, J=0.976 Hz, J=3.90 Hz), 6.91 (dd, 1H, J=3.90 Hz, J=4.88 Hz), 7.10 (dd, 1H, H=0.976 Hz, J=4.88 Hz).

### Synthesis of 2-bromo-5-octylthiophene

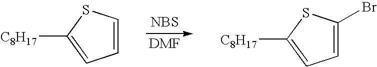

2-Octylthiophene (96.1 g. 0.489 mol) and dehydrated N,N-dimethylformamide (hereinafter referred to as “DMF”) (300 ml) were placed in a 1000-ml three-necked flask equipped with a 200-ml dropping funnel and a reflux tube, and a solution of N-bromosuccinimide (hereinafter referred to as “NBS”) (87.1 g, 0.489 mol) in DMF (200 ml) was added dropwise thereto at room temperature in an argon gas stream over a period of about one hr. After the completion of dropwise addition, the mixture was stirred with heating at 100° C. for about 2 hr. After the completion of the reaction, water (300 ml) was added to the reaction solution, and the organic layer was extracted with diethyl ether, was dried over sodium sulfate, and was applied to column chromatography (n-hexane) to give an objective compound 2-bromo-5-octylthiophene as a yellow liquid (125.4 g, yield 93.2%). An NMR spectrum of the compound thus obtained was measured at room temperature with an NMR spectrometer (model JNM-LA400W, manufactured by Japan Electric Optical Laboratory). ¹H-NMR (CDCl₃, TMS/ppm): 0.88 (t, 3H, J=6.83 Hz), 1.28 (m, 10H), 1.60 (m, 2H), 2.73 (t, 2H, J=7.32 Hz), 6.52 (d, 1H, J=3.90 Hz), 6.83 (d, 1H, J=3.90 Hz).

### Synthesis of 2-(trimethylsily)ethynyl-5-octylthiophene

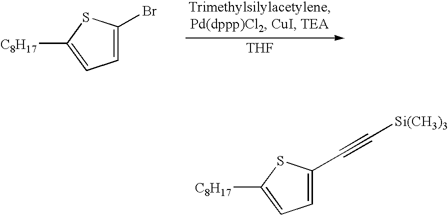

2-Bromo-5-octylthiophene (40.0 g, 0.145 mol) prepared above, trimethylsilyacetylene (14.3 g, 0.145 mol), bis(triphenylphosphine)palladium(II) dichloride (2.0 g, 2.90 mmol), copper(I) iodide (550 mg, 2.90 mmol), triethylamine (90 ml), and THF (300 ml) were placed in a 1000-ml flask equipped with a reflux tube, and the mixture was refluxed in an argon gas stream over a period of about 6 hr. After the completion of the reaction, water (200 ml) was added to the reaction solution, and the organic layer was extracted with diethyl ether, was dried over sodium sulfate, and was applied to column chromatography (n-hexane) to give an objective compound 2-(trimethylsily)ethynyl-5-octylthiophene as a light yellow liquid (42.4 g, yield 100%). An NMR spectrum of the compound thus obtained was measured at room temperature with an NMR spectrometer (model JNM-LA400W, manufactured by Japan Electric Optical Laboratory). ¹H-NMR (CDCl₃, TMS/ppm): 0.231 (s, 9H), 0.88 (t, 3H, J=6.83 Hz), 1.28 (m, 10H), 1.64 (m, 2H), 2.75 (t, 2H, J=7.32 Hz), 6.60 (d, 1H, J=3.42 Hz), 7.04 (d, 1H, J=3.42 Hz).

### Synthesis of 2-ethynyl-5-octylthiophene

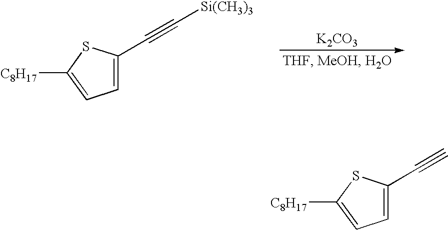

2-(Trimethylsily)ethynyl-5-octylthiophene (44.8 g, 0.153 mol) prepared above, potassium carbonate (30.0 g), water (50 ml), THF (400 ml), and methanol (100 ml) were placed in a 1000-ml flask equipped with a reflux tube, and the mixture was refluxed in an argon gas stream over a period of about 6 hr. After the completion of the reaction, water (200 ml) was added to the reaction solution, and the organic layer was extracted with diethyl ether, was dried over sodium sulfate, and was applied to column chromatography (n-hexane) to give an objective compound 2-ethynyl-5-octylthiophene as a light yellow liquid (33.2 g, yield 98.5%). An NMR spectrum of the compound thus obtained was measured at room temperature with an NMR spectrometer (model JNM-LA400W, manufactured by Japan Electric Optical Laboratory). ¹H-NMR (CDCl₃, TMS/ppm): 0.88 (t, 3H, J=6.83 Hz), 1.29 (m, 10H), 1.64 (m, 2H), 2.76 (t, 2H, J=7.32 Hz), 3.28 (s, 1H), 6.63 (dd, 1H, J=0.976 Hz, J=3.42 Hz), 7.09 (d, 1H, J=3.42 Hz)

### Synthesis of 2,5-bis(5-octyl-2-thienylethynyl)-thiophene (8T-yne-T-yne-T8)

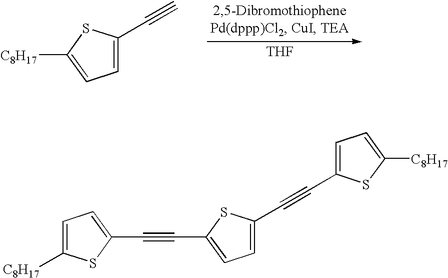

2-Ethynyl-5-octylthiophene (11.2 g, 50.8 mmol) prepared above, 2,5-dibromothiophene (6.0 g, 24.8 mmol), bis(triphenylphosphine)palladium(II) dichloride (870 mg, 1.24 mmol), copper(I) iodide (240 mg, 1.24 mmol), triethylamine (20 ml), and THF (100 ml) were placed in a 500-ml flask equipped with a reflux tube, and the mixture was refluxed in an argon gas stream over a period of about 6 hr. After the completion of the reaction, water (200 ml) was added to the reaction solution, and the organic layer was extracted with chloroform, was dried over sodium sulfate, and was applied to column chromatography (n-hexane) to give an objective compound 2,5-bis(5-octyl-2-thienylethynyl)-thiophene (8T-yne-T-yne-T8) as a light yellow powder (3.6 g, yield 27.9%). An NMR spectrum of the compound thus obtained was measured at room temperature with an NMR spectrometer (model JNM-LA400W, manufactured by Japan Electric Optical Laboratory). ¹H-NMR (CDCl₃, TMS/ppm): 0.88 (t, 6H, J=6.83 Hz), 1.30 (m, 20H), 1.67 (m, 4H), 2.79 (t, 4H, J=7.32 Hz), 6.68 (d, 2H, J=3.90 Hz), 7.10 (s, 2H), 7.11 (d, 2H, J=3.90 Hz).

### Synthesis of 5,5′-bis(5-octyl-2-thienylethynyl)-2,2′-bithiophene (8T-yne-TT-yne-T8)

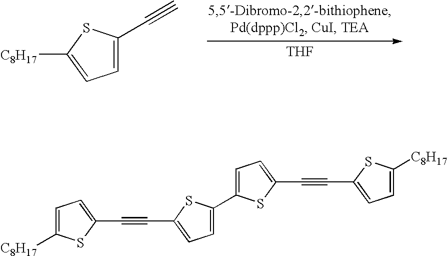

2-Ethynyl-5-octylthiophene (11.8 g, 53.5 mmol) prepared above, 5,5′-dibromo-2,2′-bithiophene (8.46 g, 26.1 mmol), bis(triphenylphosphine)palladium(II) dichloride (917 mg, 1.31 mmol), copper(I) iodide (250 mg, 1.31 mmol), triethylamine (30 ml), and THF (100 ml) were placed in a 500-ml flask equipped with a reflux tube, and the mixture was refluxed in an argon gas stream over a period of about 6 hr. After the completion of the reaction, water (200 ml) was added to the reaction solution, and the organic layer was extracted with chloroform, was dried over sodium sulfate, and was applied to column chromatography (n-hexane) to give an objective compound 5,5′-bis(5-octyl-2-thienylethynyl)-2,2′-bithiophene (8T-yne-TT-yne-T8) as a light yellow powder (5.9 g, yield 37.6%). An NMR spectrum of the compound thus obtained was measured at room temperature with an NMR spectrometer (model JNM-LA400W, manufactured by Japan Electric Optical Laboratory). ¹H-NMR (CDCl₃, TMS/ppm): 0.88 (t, 6H, J=6.83 Hz), 1.30 (m, 20H), 1.66 (m, 4H), 2.79 (t, 4H, J=7.32 Hz), 6.68 (d, 2H, J=3.90 Hz), 7.06 (d, 2H, J=3.90 Hz), 7.10 (d, 2H, J=3.90 Hz), 7.13 (d, 2H, J=3.90 Hz).

### Synthesis of 5,5″-dibromo-2,2′:5′,2″-terthiophene

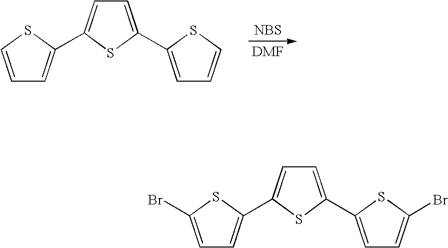

2,2′:5′,2″-Terthiophene (5.20 g, 20.9 mmol) and DMF (200 ml) were placed in a 500-ml three-necked flask equipped with a 100-ml dropping funnel and a reflux tube, and a solution of NBS (7.63 g, 42.9 mmol) in DMF (100 ml) was added dropwise thereto at room temperature in an argon stream over a period of about one hr. After the completion of the dropwise addition, the mixture was stirred with heating at 100° C. for about 2 hr. After the completion of the reaction, the reaction solution was poured into water (1000 ml), and the resultant yellow powder was collected by filtration and was dried in vacuo to give an objective compound 5,5″-dibromo-2,2′:5′,2″-terthiophene (8.56 g, yield 100%). An NMR spectrum of the compound thus obtained was measured at room temperature with an NMR spectrometer (model JNM-LA400W, manufactured by Japan Electric Optical Laboratory). ¹H-NMR (CDCl₃, TMS/ppm): 6.90 (d, 2H, J=3.90 Hz), 6.97 (d, 2H, J=3.90 Hz), 6.99 (s, 2H).

### Synthesis of 5,5″-bis(5-octyl-2-thienylethynyl)-2,2′:5,2″-terthiophene (8T-yne-TTP-yne-T8)>

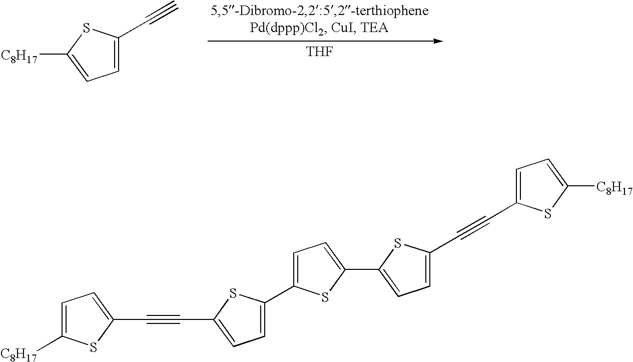

5,5″-Dibromo-2,2′:5′,2″-terthiophene (16.0 g, 39.5 mmol) prepared above, 2-ethynyl-5-octylthiophene (26.1 g, 119 mmol), and bis(triphenylphosphine)palladium(II) dichloride (737 mg, 1.05 mmol), copper(I) iodide (200 mg, 1.05 mmol), triethylamine (30 ml), and toluene (100 ml) were placed in a 500-ml flask equipped with a reflux tube, and the mixture was refluxed in an argon gas stream over a period of about 6 hr. After the completion of the reaction, water (200 ml) was added to the reaction solution, and the organic layer was extracted with chloroform, was dried over sodium sulfate, and was applied to column chromatography (n-hexane) to give an objective compound 5,5′-bis(5-octyl-2-thienylethynyl)-2,2′:5′,2″-terthiophene (8T-yne-TTP-yne-T8) (12.5 g, yield 46.3%). An NMR spectrum of the compound thus obtained was measured at room temperature with an NMR spectrometer (model JNM-LA400W, manufactured by Japan Electric Optical Laboratory). ¹H-NMR (CDCl₃, TMS/ppm): 0.89 (t, 6H, J=6.83 Hz), 1.36 (m, 20H), 1.66 (m, 4H), 2.80 (t, 4H, J=7.32 Hz), 6.68 (d, 2H, J=3.90 Hz), 7.05 (d, 2H J=3.90 Hz), 7.09 (s, 2H), 7.10 (d, 2H, J=3.90 Hz), 7.14 (d, 2H, J=3.90 Hz).

<Preparation of FET Element>

A wafer purchased from ELECTRONICS AND MATERIALS CORPORATION LIMITED was used in a test device. This wafer is an n-doped silicon wafer with a silicon oxide layer having a thickness of about 3000 angstroms (300 nm) thermally produced thereon. The wafer functioned as a gate electrode while the silicon oxide layer functioned as a gate dielectric material, and the electrostatic capacitance was about 11 nF/cm² (nanofarad/square centimeter). This wafer was immersed in a 0.1 M dehydrated toluene solution of phenyltrichlorosilane at 60° C. for 20 min. Next, this wafer was washed with toluene, and the remaining liquid was removed by a nitrogen air gun, followed by drying at 100° C. for one hr.

Next, gold source and drain electrodes were vacuum deposited onto the silicon oxide dielectric layer through a shadow mask with varied channel length and width. Thus, a series of transistor electrodes having various sizes were prepared. Thereafter, this wafer was heated to 60° C., and an organic semiconductor layer was formed by spin coating at a solution temperature of 60° C. at a speed of 2000 rpm for about 10 sec. The solution for the formation of the organic semiconductor layer was prepared by dissolving 1.0% by weight of 5,5″-bis(5-octyl-2-thienylethynyl)-2,2′:5′,2″-terthiophene (8T-yne-TTP-yne-T8) prepared above in toluene. These procedures were carried out under ambient conditions, and any measure for preventing the exposure of the material and apparatus to ambient oxygen, moisture, or light was not taken.

FET properties were evaluated by 237 HIGH VOLTAGE SOURCE MEASURE UNIT, manufactured by KEITHLEY. The carrier mobility (μ) was calculated based on data in a saturation region (gate voltage V_{G}<source-drain voltage VSD) by the following equation (1). In equation (1), I_{SD} represents drain current in the saturation region, W and L represent the width and length in the semiconductor channel, respectively, Cᵢ represents the electrostatic capacitance per unit area of the gate dielectric layer, and V_{G} and V_{T} represent gate voltage and threshold voltage, respectively. VT in this apparatus was determined from the relationship between the square root of I_{SD} in the saturation region and V_{G} in the apparatus determined from the measured data by extrapolating I_{SD}=0. The current on/off ratio is the ratio between saturation source/drain current at a gate voltage V_{G} equal to or higher than the drain voltage V_{D}, and source/drain current at a gate voltage V_{G} of zero.

I_{SD}=Cᵢμ(W/2L)(V_{G}−V_{T})² (1)

The average property value obtained from five or more transistors having a size of W (width)=1200 μm and L (length)=50 μm was hole mobility=1.0×10⁻² cm²/Vs and current on/off ratio=10⁴ (Vds=−80V). This high on/off ratio suggests that the polymer material is less likely to undergo oxidation and thus is highly stable in the atmosphere and exhibits good process properties.

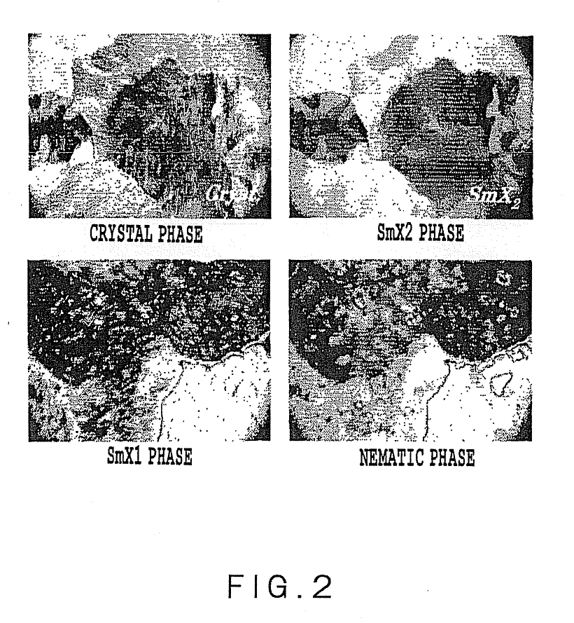

FIG. 2 shows the results of observation of texture by a polarizing microscope using a glass cell into which 8T-yne-TTP-yne-T8 has been poured. In the preparation of the FET element, the phase transition temperature between the crystal phase and the SmX2 phase in 8T-yne-TTP-yne-T8 per se is 77.2° C. Since, however, the solution of 8T-yne-TTP-yne-T8 in toluene has a lowered phase transition temperature due to the mixing effect, a coating film of 8-yne-TTP-yne-8 in a liquid crystalline solution (mixed liquid crystal state) could be formed even by heating at about 60° C.

### Example 2

In Example 2, organic semiconductor materials represented by chemical formula 1, wherein R1 and R2 represents a C8 identical straight chain alkyl group and n1 is 3 and 4, were prepared.

### Synthesis of 5,5″-bis(decyl-2-yne)-2,2′:5′,2″-terthiophene (8-yne-TTP-yne-8)

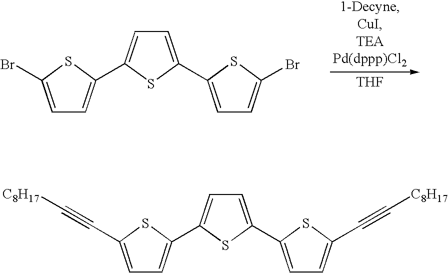

5,5′-Dibromo-2,2′:5′,2″-terthiophene (4.5 g, 11.1 mmol), 1-decyne (3.37 g, 24.4 mmol), bis(triphenylphosphine)palladium(II) dichloride (390 mg, 0.550 mmol), copper(I) iodide (105 mg, 0.550 mmol), triethylamine (20 ml), and THF (100 ml) were placed in a 1000-ml flask equipped with a reflux tube, and the mixture was refluxed in an argon gas stream over a period of about 6 hr. After the completion of the reaction, water (200 ml) was added to the reaction solution, and the organic layer was extracted with chloroform, was dried over sodium sulfate, and was applied to column chromatography (n-hexane) to give an objective compound 5,5″-bis(decyl-2-yne)-2,2′:5′2″-terthiophene (8-yne-TTP-yne-8) as a light yellow powder (6.2 g, yield 56.8%). An NMR spectrum of the compound thus obtained was measured at room temperature with an NMR spectrometer (model JNM-LA400W, manufactured by Japan Electric Optical Laboratory). ¹H-NMR (CDCl₃, TMS/ppm): 0.89 (t, 6H, J=6.83 Hz), 1.38 (overlapped peaks, 20H), 1.59 (m, 4H), 2.43 (t, 4H, J=6.83 Hz), 6.90 (d, 2H, J=3.42 Hz), 7.00 (d, 2H, J=3.42 Hz), 7.03 (s, 2H).

### Synthesis of 5,5″-dibromo-2,2′:5′,2″:5″,2′″-quaterthiophene

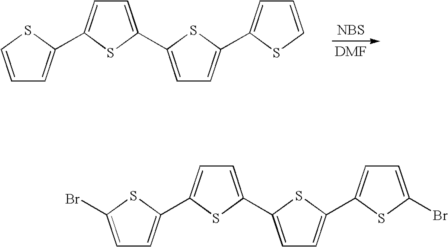

2,2′:5′,2″:5″,2′″-Quaterthiophene (20.0 g, 60.5 mmol) and DMF (500 ml) were placed in a 1000-ml three-necked flask equipped with a 100-ml dropping funnel and a reflux tube, and a solution of NBS (22.1 g, 124 mmol) in DMF (100 ml) was added dropwise thereto at 120° C. in an argon stream over a period of about one hr. After the completion of the dropwise addition, the mixture was stirred with heating at 120° C. for about 2 hr. After the completion of the reaction, the reaction solution was poured into water (1000 ml), and the resultant yellowish brown powder was collected by filtration and was dried in vacuo to give an objective compound 5,5″-dibromo-2,2′:5′,2″:5″,2′″-quaterthiophene (29.5 g, yield 100%). The compound thus obtained was subjected to mass analysis (GCMS-QP5000 manufactured by Shimadzu Seisakusho Ltd.). EI-MS: m/e=487.90 (C₁₆H₈Br₂S₄, M+, 100%).

### Synthesis of 5,5-m-bis(decyl-2-yne)-2,2′:5′,2″:5″,2′″-quarter thiophene (8-yne-QT-yne-8)

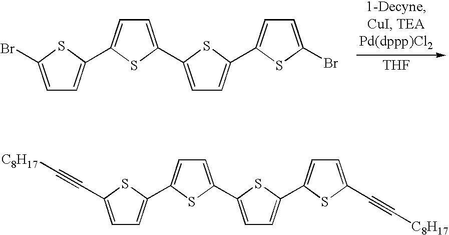

5,5′″-Dibromo-2,2′:5′,2″:5″,2′″-quaterthiophene (10.0 g, 20.5 mmol), 1-decyne (11.3 g, 81.9 mmol), bis(triphenylphosphine)palladium(II) dichloride (720 mg, 1.02 mmol), copper(I) iodide (194 mg, 1.02 mmol), triethylamine (100 ml), and toluene (200 ml) were placed in a 1000-ml flask equipped with a reflux tube, and the mixture was refluxed in an argon gas stream over a period of about 6 hr. After the completion of the reaction, water (200 ml) was added to the reaction solution, and the organic layer was extracted with chloroform, was dried over sodium sulfate, and was applied to column chromatography (n-hexane: CHCl₃=9:1) to give an objective compound, 5′″-bis(decyl-2-yne)-2,2′:5′,2″:5″,2′″-quaterthiophene (8-yne-QT-yne-8) as a yellow powder (7.2 g, yield 58.1%). An NMR spectrum of the compound thus obtained was measured at room temperature with an NMR spectrometer (model JNM-LA400W, manufactured by Japan Electric Optical Laboratory). ¹H-NMR (CDCl₃, TMS/ppm): 0.89 (t, 6H, J=6.83 Hz), 1.36 (overlapped peaks, 20H), 1.60 (m, 4H), 2.44 (t, 4H, J=7.32 Hz), 7.02 (m, 8H).

<Preparation of FET Element>

A wafer purchased from ELECTRONICS AND MATERIALS CORPORATION LIMITED as used in Example 1 was used in a test device. A source electrode and a drain electrode were vacuum deposited in the order of chromium (5 nm) and gold (50 nm) in that order on this wafer through shadow masks with various channel lengths and widths. A series of transistor electrodes having various sizes were prepared. Next, this wafer was immersed in a 0.1 M dehydrated toluene solution of phenyltrichlorosilane at 60° C. for 20 min. Next, this wafer was washed with toluene, and the remaining liquid was removed by a nitrogen air gun, followed by drying at 100° C. for one hr.

Next, this wafer was heated to 90° C., and an organic semiconductor layer was formed by spin coating at a solution temperature of 90° C. at a speed of 2000 rpm for about 10 sec. The solution for the formation of the organic semiconductor layer was prepared by dissolving 1.0% by weight of 5,5′″-bis(decyl-2-yne)-2,2′:5′,2″:5″,2′″-quaterthiophene (8-yne-QT-yne-8) in xylene. These procedures were carried out under ambient conditions, and any measure for preventing the exposure of the material and apparatus to ambient oxygen, moisture, or light was not taken.

FET properties were evaluated by 237 HIGH VOLTAGE SOURCE MEASURE UNIT, manufactured by KEITHLEY as used in Example 1. The carrier mobility (μ) was calculated based on data in a saturation region (gate voltage V_{G}<source-drain voltage VSD) by the above equation (1). The average property value obtained from five or more transistors having a size of W (width)=1200 μm and L (length)=25 μm was hole mobility=1.7×10⁻² cm²/Vs and current on/off ratio=10⁵ (V_{ds}=−80V). This high on/off ratio suggests that the polymer material is less likely to undergo oxidation and thus is highly stable in the atmosphere and exhibits good process properties.

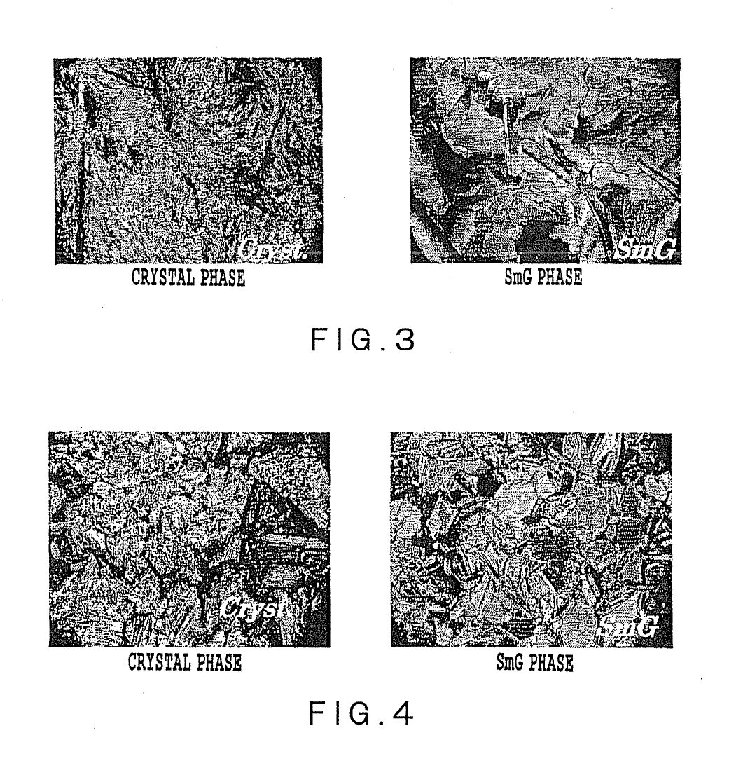

FIG. 3 shows the results of observation of texture by a polarizing microscope (BH2-UMA, manufactured by Olympus Corporation) and a heating stage (FP82HT and FP80HT, manufactured by METTLER-TOLEDO K.K.) using a glass cell into which 8-yne-TTP-yne-8 has been poured. FIG. 4 shows the results of observation of texture by a polarizing microscope (BH2-UMA, manufactured by Olympus Corporation) and a heating stage (FP82HT and FP80HT, manufactured by METTLER-TOLEDO K.K.) using a glass cell into which 8-yne-QT-yne-8 has been poured. In the preparation of the FET element, the phase transition temperature between the crystal phase and the SmG phase in 8-yne-QT-yne-8 per se is 101.2° C. Since, however, the solution of 8-yne-QT-yne-8 in xylene has a lowered phase transition temperature due to the mixing effect, a coating film of 8-yne-QT-yne-8 in a liquid crystalline solution (mixed liquid crystal state) could be formed even by heating at about 90° C.

1. An organic semiconductor material, which is represented by the following chemical formula wherein R3 and R4 represent an identical alkyl group having 1 to 20 carbon atoms and n2 is 1 to 4:

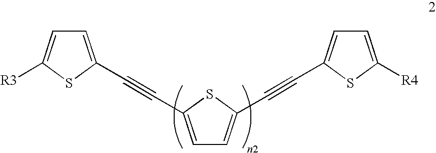

2. An organic semiconductor structure comprising an organic semiconductor layer comprising an organic semiconductor material according to claim 1.

3. An organic semiconductor device comprising at least a substrate, a gate electrode, a gate insulating layer, an organic semiconductor layer, a drain electrode, and a source electrode, said organic semiconductor layer comprising an organic semiconductor material according to claim 1.
